(19) United States
(12) Patent Application Publication
(10) Pub. No.: US 2017/0164887 A1
(43) Pub. Date: Jun. 15, 2017

(54) APPARATUS AND METHOD FOR ESTIMATING CHANGES TO HUMAN HEALTH BASED ON MONITORING HAIR PARAMETERS

(71) Applicant: INTEL CORPORATION, Santa Clara, CA (US)

(72) Inventors: Rita Chattopadhyay, Chandler, AZ (US); Akanksha Chattopadhyay, Chandler, AZ (US); William C. DeLeeuw, Beaverton, OR (US)

(21) Appl. No.: 14/967,179

(22) Filed: Dec. 11, 2015

(51) Int. Cl. A61B 5/00; G01B 11/06; G01N 33/68; A42B 3/04; A61B 5/01; A46B 9/02; A46B 15/00; A45D 8/34; G01N 27/22; H04N 7/18

(52) U.S. Cl. CPC A61B 5/448; G01N 27/225; G01B 11/06; G01N 33/68; H04N 7/185; A61B 5/01; A46B 9/023; A46B 15/0022; A45D 8/34; A42B 3/044; A42B 3/042

(57) ABSTRACT

Described is an apparatus comprising: an ensemble of sensors including a moisture sensor to sense moisture content of hair, the moisture sensor positioned in a first housing to be in contact with the hair; and a processor to receive data associated with sensed moisture content and to transmit information related to the received data to another device, the processor positioned in a second housing different from the first housing.

### BACKGROUND

Health of human hair and scalp may be a major clue to a wide variety of health conditions. For example, human hair responds to stress (e.g., physical stress and psychological stress). A whole host of internal conditions affect the health of human hair, and as such hair related parameters such as color, thickness, texture, etc., may indicate health disorders such as such stress, fatigue, weight gain, diabetes, slow heart rate, age, lack of essential nutrients, bone growth, etc.

### BRIEF DESCRIPTION OF THE DRAWINGS

The embodiments of the disclosure will be understood more fully from the detailed description given below and from the accompanying drawings of various embodiments of the disclosure, which, however, should not be taken to limit the disclosure to the specific embodiments, but are for explanation and understanding only.

FIG. 1 illustrates an ensemble of wearable devices including a device with one or more sensors used for estimating changes to human health by monitoring hair parameters, according to some embodiments of the disclosure.

FIG. 2 illustrates a wearable device (e.g., a hair band) with sensors used for estimating changes to human health, according to some embodiments of the disclosure.

FIG. 3A illustrates a top view of a hair brush including one or more sensors used for estimating changes to human health, according to some embodiments of the disclosure.

FIG. 3B illustrates a cross-sectional view of a hair brush including one or more sensors used for estimating changes to human health, according to some embodiments of the disclosure.

FIG. 4 illustrates a high level architecture of a wearable device, according to some embodiments of the disclosure.

FIG. 5 illustrates a flowchart of a method for monitoring hair parameters for estimating changes to human health, according to some embodiments of the disclosure.

FIG. 6 illustrates a part of system of FIG. 4 with a machine readable storage medium (or media) having instructions for hair and human health analysis, according to some embodiments of the disclosure.

FIG. 7 illustrates a smart device or a computer system or a SoC (System-on-Chip) with one or more sensors used for estimating changes to human health, according to some embodiments.

### DETAILED DESCRIPTION

Various embodiments describe methods and apparatuses for estimating human health and/or estimating changes to human health by monitoring parameters related to hair. Hair may be an indicator of human health and may be used for estimating changes to human health. For example, several hair related parameters such as; hair color, texture, porosity, thickness, moisture content, oil, besides scalp conditions, etc. may be used to estimate changes to human health conditions such as stress level, fatigue, weight gain, slow heart rate, age, lack of essential nutrients (e.g., eating disorder), diabetes, and bone growth. Various embodiments employ one or more sensors for monitoring hair parameters. These sensors include moisture sensors, chemical sensors, temperature sensors, thickness sensors, cameras, etc., in accordance with some embodiments.

In some embodiments, these one or more sensors are integrated within hair accessories such as hair bands, hair ties, hair pins, hair bows, pairs of spectacles, caps, hats, etc. In some embodiments, the apparatus is a sensor mounted hair accessory for estimating human health. In some embodiments, the one or more sensors monitor vital parameters related to hair such as moisture content or dryness of the hair, hair texture, thickness etc., and as such the apparatus of various embodiments can be used to monitor the health of the hair. In some embodiments, the sensed parameters are collected by an integrated processor or microcontroller and transmitted wirelessly to another device (e.g., cloud, smartphone, laptop, personal computer, etc.) where the sensed parameters are analyzed to estimate the health of the hair.

There are several products in the market to monitor human health based on wearable sensors, such as bulky watches, gloves, helmets etc. which monitor blood pressure, body temperature, pulse rate, electrocardiogram, etc. Most of these devices are very conspicuous and non-cosmetic, having very poor aesthetics. These devices may sometimes even impair normal functioning of the body.

In some embodiments, the one or more sensors are integrated in wearable accessories such as hair bands, hair clips, hair ties, etc. which have cosmetic value besides being able to monitor and/or estimate human health. There wearable accessories with integrated sensors, transmitters, and processor(s) are referred to as wearable devices. In some embodiments, the wearable devices are hands-free and may not cause any impediment to a user. And since the sensors are embedded in the hair accessories, the sensors are inconspicuous besides being noninvasive, in accordance with some embodiments. In some embodiments, another device (e.g., cloud) receives the collected data from the sensors and performs analytical analysis on the data.

In the following description, numerous details are discussed to provide a more thorough explanation of embodiments of the present disclosure. It will be apparent, however, to one skilled in the art, that embodiments of the present disclosure may be practiced without these specific details. In other instances, well-known structures and devices are shown in block diagram form, rather than in detail, in order to avoid obscuring embodiments of the present disclosure.

Note that in the corresponding drawings of the embodiments, signals are represented with lines. Some lines may be thicker, to indicate more constituent signal paths, and/or have arrows at one or more ends, to indicate primary information flow direction. Such indications are not intended to be limiting. Rather, the lines are used in connection with one or more exemplary embodiments to facilitate easier understanding of a circuit or a logical unit. Any represented signal, as dictated by design needs or preferences, may actually comprise one or more signals that may travel in either direction and may be implemented with any suitable type of signal scheme.

Throughout the specification, and in the claims, the term “connected” means a direct connection, such as electrical, mechanical, or magnetic connection between the things that are connected, without any intermediary devices. The term “coupled” means a direct or indirect connection, such as a direct electrical, mechanical, or magnetic connection between the things that are connected or an indirect connection, through one or more passive or active intermediary devices. The term “circuit” or “module” may refer to one or more passive and/or active components that are arranged to cooperate with one another to provide a desired function. The term “signal” may refer to at least one current signal, voltage signal, magnetic signal, or data/clock signal. The meaning of “a,” “an,” and “the” include plural references. The meaning of “in” includes “in” and “on.”

The term “scaling” generally refers to converting a design (schematic and layout) from one process technology to another process technology and subsequently being reduced in layout area. The term “scaling” generally also refers to downsizing layout and devices within the same technology node. The term “scaling” may also refer to adjusting (e.g., slowing down or speeding up—i.e. scaling down, or scaling up respectively) of a signal frequency relative to another parameter, for example, power supply level. The terms “substantially,” “close,” “approximately,” “near,” and “about,” generally refer to being within +/−10% of a target value.

Unless otherwise specified the use of the ordinal adjectives “first,” “second,” and “third,” etc., to describe a common object, merely indicate that different instances of like objects are being referred to, and are not intended to imply that the objects so described must be in a given sequence, either temporally, spatially, in ranking or in any other manner.

For the purposes of the present disclosure, phrases “A and/or B” and “A or B” mean (A), (B), or (A and B). For the purposes of the present disclosure, the phrase “A, B, and/or C” means (A), (B), (C), (A and B), (A and C), (B and C), or (A, B and C). The terms “left,” “right,” “front,” “back,” “top,” “bottom,” “over,” “under,” and the like in the description and in the claims, if any, are used for descriptive purposes and not necessarily for describing permanent relative positions.

For purposes of the embodiments, the transistors in various circuits and logic blocks described here are metal oxide semiconductor (MOS) transistors or their derivatives, where the MOS transistors include drain, source, gate, and bulk terminals. The transistors and/or the MOS transistor derivatives also include Tri-Gate and FinFET transistors, Gate All Around Cylindrical Transistors, Tunneling FET (TFET), Square Wire, or Rectangular Ribbon Transistors, or other devices implementing transistor functionality like carbon nanotubes or spintronic devices. MOSFET symmetrical source and drain terminals i.e., are identical terminals and are interchangeably used here. A TFET device, on the other hand, has asymmetric Source and Drain terminals. Those skilled in the art will appreciate that other transistors, for example, Bi-polar junction transistors—BJT PNP/NPN, BiCMOS, CMOS, etc., may be used without departing from the scope of the disclosure.

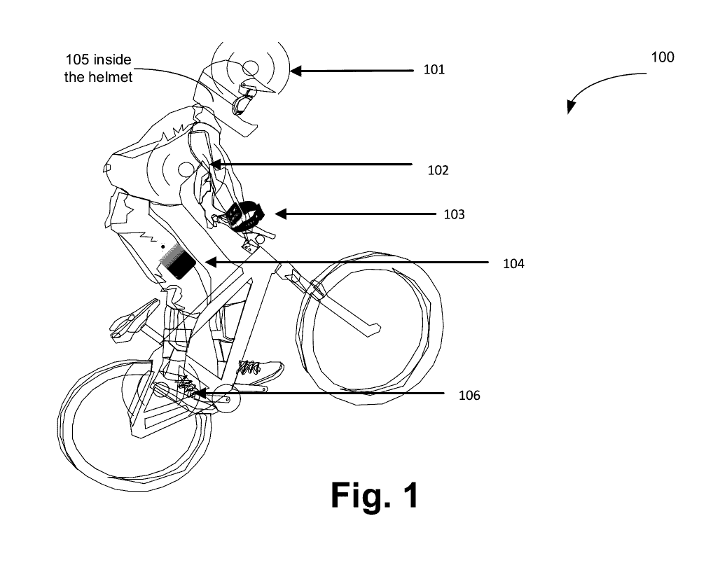

FIG. 1 illustrates ensemble 100 of wearable devices including a device with one or more sensors used for estimating human health using monitored hair parameters, according to some embodiments of the disclosure. In this example, ensemble 100 is on a person and his/her ride (here, a bicycle). However, the embodiments are not limited to such. Other scenarios of wearable devices and their usage may work with the various embodiments. For example, sensors can be embedded in the googles worn by the person such that the sensors are in direct contact with the hair of the person.

In some embodiments, the sensor node(s) are part of a wearable device. The term “wearable device” (or wearable computing device) generally refers to a device coupled to a person. For example, devices or accessories (such as sensors, cameras, speakers, microphones (mic), smartphones, smart watches, hair bands, hats, helmet, hair pins, pairs of spectacles, hair brush, comb, etc.) which are directly attached on a person, on the person's clothing, or on a person's accessories are within the scope of wearable devices.

In some examples, wearable computing devices may be powered by a main power supply such as an AC/DC (Alternating Current and/or Direct Current) power outlet. In some examples, wearable computing devices may be powered by a battery. In some examples, wearable computing devices may be powered by a specialized external source based on Near Field Communication (NFC). The specialized external source may provide an electromagnetic field that may be harvested by circuitry at the wearable computing device. Another way to power the wearable computing device is electromagnetic field associated with wireless communication, for example, WLAN transmissions. WLAN transmissions use far field radio communications that have a far greater range to power a wearable computing device than NFC transmission. WLAN transmissions are commonly used for wireless communications with most types of terminal computing devices.

For example, the WLAN transmissions may be used in accordance with one or more WLAN standards based on Carrier Sense Multiple Access with Collision Detection (CSMA/CD) such as those promulgated by the Institute of Electrical Engineers (IEEE). These WLAN standards may be based on CSMA/CD wireless technologies such as Wi-Fi™ and may include Ethernet wireless standards (including progenies and variants) associated with the IEEE 802.11-2012 Standard for Information technology—Telecommunications and information exchange between systems—Local and metropolitan area networks—Specific requirements Part 11: WLAN Media Access Controller (MAC) and Physical Layer (PHY) Specifications, published March 2012, and/or later versions of this standard (“IEEE 802.11”).

In some embodiments, ensemble 100 of wearable devices includes device 101 (e.g., camera, microphone, etc.) mounted on a helmet, device 102 (e.g., blood pressure sensor, etc.) strapped on the person's arm, device 103 (e.g., a smart watch that can function as a terminal controller or a device to be controlled), device 104 (e.g., a smart phone and/or tablet in a pocket of the person's clothing), and device 106 (e.g., an accelerometer to measure paddling speed). In some embodiments, ensemble 100 of wearable devices has the capability to communicate by wireless energy harvesting mechanisms or other types of wireless transmission mechanisms.

In some embodiments, the helmet includes an inner covering 105 that directly couples to a person's head. In some embodiments, inner covering 105 includes processing logic, moisture sensors, camera and/or camera lenses, light sources, chemical sensors, thickness sensors, oil sensors, analog-to-digital converter (ADC), temperature sensor, bus, micro-controller or processor, antenna, and battery pack. In some embodiments, when the helmet is taken off and placed on its stand (not shown), the battery pack of inner covering 105 charges.

In some embodiments, the one or more sensors of inner covering 105 sense different parameters of the person's hair, which are estimators of the health of the person riding the bike. Various hair parameters include: dryness or moisture content of the hair, thickness of hair strands, scaly or crusty patches on the person's scalp, number of hair, hair brittleness, yellowish flakes on the hair, gray hair, etc.

Many factors can lead to over-dry hair, including hair dyes, hair blowers, and swimming in chlorinated water. But a significant change in texture that leaves hair feeling finer, with less body or mass, can be an indicator of an underactive thyroid, known as hypothyroidism. In some embodiments, the one or more moisture sensors in the inner covering 105 are used for measuring the moisture content of the hair. In some embodiments, one or more thickness sensors can be used to measure the diameter of the hair strands. In some embodiments, the camera and associated light source can be used to take a picture of the hair to estimate the thickness of the hair strands.

When a thick crust forms on the scalp, this may indicate psoriasis, which can be distinguished from other dandruff-like skin conditions by the presence of a thickening scab-like surface. Psoriasis is the most common of all the autoimmune diseases and occurs when the skin goes into overdrive, sending out faulty signals that speed up the turnover and growth of skin cells. In some embodiments, a thickness sensor is integrated in the inner covering 105 to sense changing thickening of the scalp. In some embodiments, the camera and associated light source can be used to take a picture of the hair to sense changing thickening of the scalp.

Another hair parameter to monitor is hair loss. It is normal to shed approximately 100 to 150 hairs a day, which may be the result of the body's natural turnover. However, when hair appears to be coming out in clumps then it may be time for concern. One common cause of hair loss may be a sudden psychological or physical stressor. Physical stressors that may lead to temporary hair loss include iron deficiency anemia and protein deficiency; these are particularly common in those who suffer from eating disorders. Diabetes may also cause hair to thin or to start to fall out suddenly. Sudden hair thinning or hair loss may be considered an early warning sign that diabetes is affecting hormone levels. A number of medications may also cause hair loss as a side effect. Hormonal changes may also cause hair to thin. Thyroid disease, especially hypothyroidism, may be one of the most common causes of hair loss. In some embodiments, a hair density sensor is integrated in the inner covering 105 to sense changing density of the hair. In some embodiments, the camera and associated light source may be used to take a picture of the hair to sense the change in density of the hair on the scalp.

Dry brittle hair that breaks off easily may also be a parameter of the hair that may indicate a person's health. Breakage may be most frequently the result of hair becoming over-brittle from chemical processing or dyeing. However, certain health conditions may also lead to brittle, fragile hair. Among these health conditions include Cushing's syndrome, hypoparathyroidism, low levels of parathyroid hormone, lack of omega-3 fatty acids.

Cushing's syndrome is a disorder of the adrenal glands that causes excess production of the hormone cortisol. Hypoparathyroidism is usually either hereditary or the result of injury to the parathyroid glands during head and neck surgery, and may also cause dry, brittle hair. Overly low levels of parathyroid hormone cause blood levels of calcium to fall and phosphorus to rise, leading to fragile dry hair, scaly skin, and more serious symptoms such as muscle cramps and even seizures. In some embodiments, the one or more moisture sensors in the inner covering 105 are used for estimating (and/or measure) the moisture content of the hair which in turn can be used to estimate one or more of the health conditions discussed above.

Another hair parameter to monitor is dandruff. Dandruff is a complicated interaction of health issues that deserve to be taken seriously. Seborrheic dermatitis is a chronic inflammatory condition of the scalp that causes skin to develop scaly patches, often in the areas where the scalp is oiliest. When the flaky skin loosens, it leaves the telltale “dandruff” flakes. In some embodiments, the camera and associated light source in inner covering 105 are used to take a picture of the hair to the scalp condition such as presence of flakes on the hair.

Hair color is also a hair parameter that may provide a window to a person's health. Stress may trigger a chain reaction that interferes with how well the hair follicle transmits melanin, the pigment that colors hair. Free radicals are hormones that may produce when under stress, and may block the signal that tells the hair follicle to absorb the melanin pigment, causing premature gray hair. In some embodiments, the camera and associated light source in inner covering 105 are used to take a picture of the hair to sense or estimate the change in hair color.

Nutritional deficiency can also be estimated by measuring the levels and comparative ratios of nutrient and toxic minerals found in hair. Hair reflects the mineral content of the body's tissues. If a mineral is either deficient or present in excess, it indicates a mineral deficiency or excess within the body, especially in conditions of malnourishment leading to protein deficiency which may be characterized by dry and light-colored hair. Similarly, zinc deficiency may cause diffuse hair loss, lighter colored hair, and eczema. Similar changes are seen in cases of fatty acid deficiency. Viral and bacterial infections and general weakness of the body might may also leave their trail in hair. In some embodiments, one or more chemical sensors in inner covering 105 are provided for analyzing and/or estimating protein and mineral content of the hair.

In some embodiments, the signals for the sensors of inner covering 105 are digitized and transmitted to a terminal device (e.g., cloud, personal computer, laptop, etc.) over Wi-Fi (or other wireless technologies) by a micro-controller. In some embodiments, software (or machine executable instructions) in the terminal device are used to quantify the digitized data to estimate hair parameters such as dryness, thickness, hair color change, scalp conditions, and mineral contents of the hair. In some embodiments, the terminal device alerts the user and/or reports to a server if one or more of these estimated hair parameters are beyond their normal limit or show any sudden change from their previous values. Further analysis may take place on remote servers, in accordance with some embodiments.

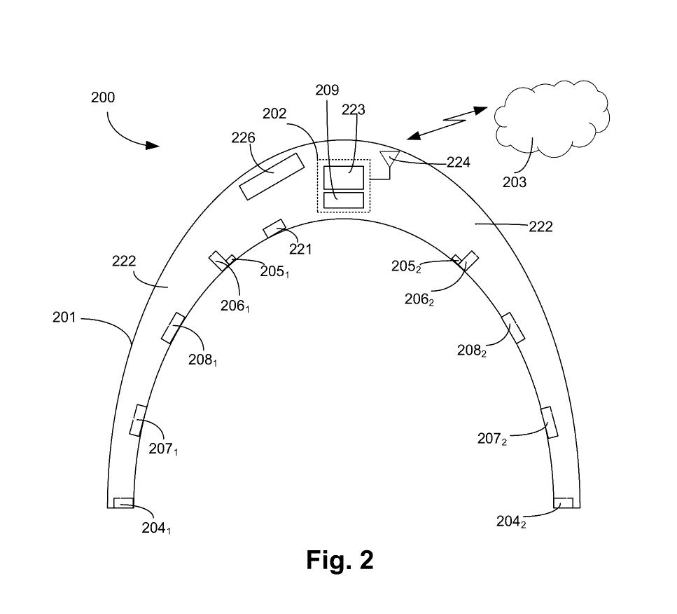

FIG. 2 illustrates a wearable device (e.g., hair band) with sensors used for estimating human health and/or changes to human health, according to some embodiments of the disclosure. FIG. 2 illustrates a system 200 with hair band 201 and a cloud 203. In some embodiments, hair band 201 comprises processing logic 202, moisture sensors 204₁₋₂, camera and/or camera lenses 205₁₋₂, light sources 206₁₋₂, chemical sensors 207₁₋₂, thickness sensors 208₁₋₂, analog-to-digital converter (ADC) 209, temperature sensor 221, bus 222, micro-controller or processor 223, antenna 224, and battery pack 226. While the embodiment of hair band 201 is illustrated with certain number of sensors and certain locations of the sensors, these number and locations can be adjusted without changing the scope of the embodiments.

In some embodiments, processing logic 202 comprises processor 223 and ADC 209. In some embodiments, ADC 209 is integrated in processor 223. In some embodiments, processor 223 is a low power micro-controller or processor such as Intel Curie® processor by Intel Corporation of Santa Clara, Calif. In other embodiments, processor(s) from other vendors may be used so long as they are low power and can be woven into a wearable fabric or accessory. In some embodiments, processor 223 includes motion sensor, Bluetooth radio, and battery charging capabilities.

In some embodiments, processor 223 includes a receiver (Rx) to receive sensor signals from the ensemble of sensors. In some embodiments, processor 223 includes a transmitter (Tx) to transmit the signals over Wi-Fi to another device (e.g., a terminal device). In some embodiments, the data is transmitted to cloud 203 which is may be any computing resource. In some embodiments, cloud 203 performs the analytics using the sensor data. In some embodiments, instead of cloud 203, data is transmitted to a terminal device such as a smartphone, tablet PC, laptop, etc. In some embodiments, processor 223 is an always-on (AON) processor that receives power from battery pack 226. In some embodiments, battery pack 226 is a rechargeable battery pack.

In some embodiments, the various sensors in hair band 201 are embedded in the fabric of hair band 201. In some embodiments, the various sensors send their signals to ADC 209 over the flexible copper conducting wires (or bus) 222 which are interleaved in the fabric or structure of hair band 201. Different connection techniques and electrical connectors for fabrics may be used as conducting wires 222. For example, Universal fabric snap fastener, fabric USB connector, stainless-steel wires woven in fabric and used as bus for electronic system, flexible electronic test module connected with embroidered conductive yarn, Petex (Sefar)—embedded copper wires with insulation varnish, etc. may be used in conjunction with and/or instead of conducting wires 222.

In some embodiments, ADC 209 converts the analog sensor signals to their digital representations. These digital signals (sampled with appropriate sampling rates) are then transmitted to microcontroller 223, for further processing to generate health indexes and wireless transmission, in accordance with some embodiments.

In some embodiments, moisture sensors 204₁₋₂ are hygrometers which are used to measure and/or estimate moisture content in the surrounding environment. In some embodiments, moisture sensors 204₁₋₂ are positioned near the tip of the hair band 201 to make sure moisture sensors 204₁₋₂ are measuring the moisture content of the hair. In other embodiments, moisture sensors 204₁₋₂ are positioned is any suitable place in hair band 201 that provides most access to the scalp to estimate moisture content of the hair on the scalp. There are generally three types of hygrometer instruments, all engineered in a slightly different way, but aim to produce a measurement and/or estimation of humidity content.

A typical hygrometer is a Capacitive Humidity Detector (CHD). CHD is designed with a polymer film sandwiched between metal conductive electrodes. The capacitive relative humidity (RH) sensor is formed by merging two conductive plates that sandwich an insulator referred to as a dielectric. The electrical capacitance is higher when the dielectric collects more water, the RH of the hair will therefore be proportional to the dielectric coefficient. In some embodiments, signals from moisture sensors 204₁₋₂ (or humidity sensors) are used to compute and/or estimate the moisture content of the hair.

In some embodiments, camera and/or camera lenses 205₁₋₂ are provided to capture the condition of the scalp and hair on a regular or continuous basis. In some embodiments, camera lenses 205₁₋₂ are macro lenses and have a short focal length which is configured to take close-up pictures of the hair and scalp. In some embodiments, camera and/or camera lenses 205₁₋₂ is a high resolution camera which is used to capture sharp images of the scalp to estimate the scalp condition. In some embodiments, camera and/or camera lenses 205₁₋₂ is used to determine flakes on the hair strands, color of the hair, density of the hair, thickness of the hair strands, etc.

In some embodiments, the video images from camera and/or camera lenses 205₁₋₂ are used to detect the color and texture of the hair. In some embodiments, the images are also used to measure and/or estimate the density of the hair, enabling a measure of hair loss, alleviating the use of a Trichometer.

In some embodiments, a light source 206₁₋₂ (e.g., a light emitting diode) is used to shine light for the camera lens to capture the image. In some embodiments, the light source is operable to provide at least one of: polarized light; ultraviolet light; or florescent light to the scalp and/or hair. In some embodiments, an actuator (e.g., part of light source 206₁₋₂) is provided to light the hair for the camera. In some embodiments, the type of light can be adjusted to get a heat map of the scalp region by camera lenses 205₁₋₂.

In some embodiments, chemical sensors 207₁₋₂ are provided for measuring and/or estimating protein and mineral contents of the hair. In some embodiments, chemical sensors 207₁₋₂ employ a “lab in a chip” technology to make it wearable or embeddable in a hair accessory such as hair band 201. In some embodiments, chemical sensors 207₁₋₂ use silicon photonics to look for a particular kind of protein. As such, different kinds and number of chemical sensors may be used for detecting particular kinds of proteins.

In some embodiments, chemical sensors 207₁₋₂ collect data to perform hair tissue mineral analysis (HTMA). HTMA is an analytical test which measures and/or estimates the mineral content of the hair. Presence or absence of some essential minerals in the human body can thus be detected using HTMA. A HTMA reveals a unique metabolic world, intracellular activity, which cannot be seen through most other tests. This provides a blueprint of the biochemistry occurring during the period of hair growth and development.

Similarly, hair mineral analysis may be used to detect and/or estimate the presence or deficiency of the following essential minerals in the human body. In some embodiments, chemical sensors 207₁₋₂ are designed to detect Zinc content in the hair and/or scalp. Zinc is involved in the production, storage and secretion of insulin and may be necessary for growth hormones.

In some embodiments, chemical sensors 207₁₋₂ are designed to detect Magnesium. Magnesium may be required for normal muscular function, especially the heart. A deficiency of Magnesium may be associated with an increased incidence of abnormal heart conditions, anxiety and nervousness.

In some embodiments, chemical sensors 207₁₋₂ are designed to detect Potassium. Potassium may be critical for normal nutrient transport into the cell. A deficiency in potassium may result in muscular weakness, mild depression and lethargy. In some embodiments, chemical sensors 207₁₋₂ are designed to detect Sodium. Excess Sodium may be associated with hypertension, but adequate amounts of sodium may be required for normal health.

In some embodiments, hair band 201 includes oil sensors (which may be a subset of chemical sensors 207₁₋₂). In some embodiments, the oil sensors are configured to differentiate between natural hair oil versus applied oil. In some embodiments, the picture captured by camera and/or camera lens 205₁₋₂ can be used to determine and/or estimate whether the scalp is producing nature oil.

In some embodiments, thickness sensors 208₁₋₂ are provided to measure thickness of the hair strands. Hair thickness or diameter of hair strands can be measured using Cross-Section Trichometer, which is a device for measuring Hair Quantity, Hair Loss, and Hair Growth. In some embodiments, thickness sensors 208₁₋₂ measure and/or estimate cross-section of hair arising from a measured cross-section of scalp. In some embodiments, thickness sensors 208₁₋₂ include a laser which shines light on the hair and estimate and/or measure the hair diameter by the scattering of the laser light. The scattering creates a diffraction pattern including a line of light and dark spots which are used to estimate the thickness of the hair. In some embodiments, temperature sensor 221 is used to provide the temperature of the scalp. Any known temperature or thermal sensor technique can be used for implementing temperature sensor 221.

In some embodiments, antenna 224 is provided to communicate with another device (e.g., cloud 203). In some embodiments, antenna 224 is one of: monopole antennas, dipole antennas, loop antennas, patch antennas, microstrip antennas, coplanar wave antennas, or other types of antennas suitable for transmission of Radio Frequency (RF) signals. In some multiple-input multiple-output (MIMO) embodiments, antenna array 224 are separated to take advantage of spatial diversity.

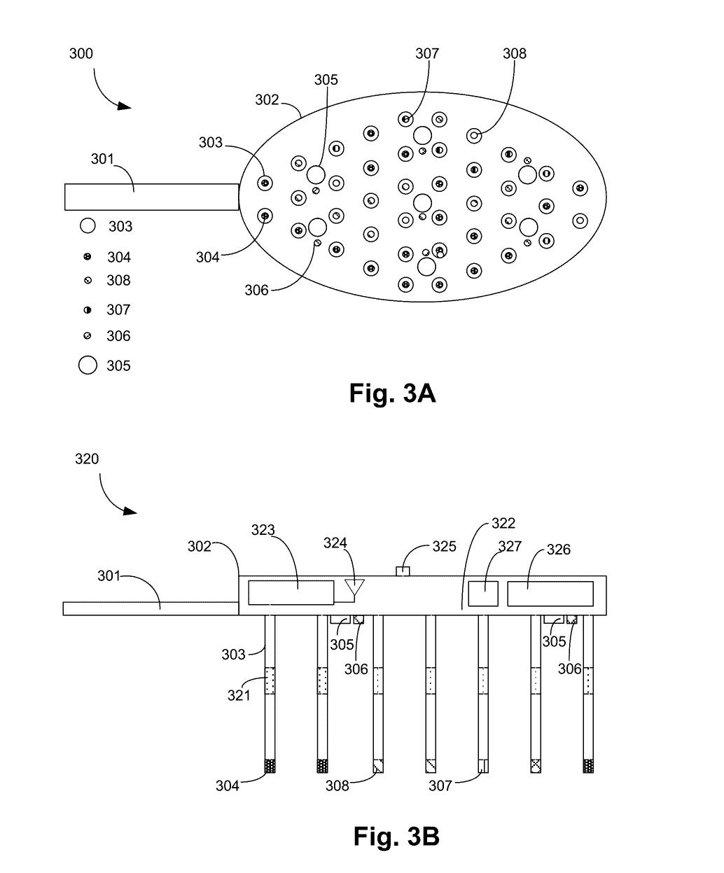

FIG. 3A illustrates top view 300 of a hair brush including one or more sensors used for estimating human health, according to some embodiments of the disclosure. It is pointed out that those elements of FIG. 3A having the same reference numbers (or names) as the elements of any other figure can operate or function in any manner similar to that described, but are not limited to such.

In some embodiments, hair brush comprises a handle 301 and a brush housing (or main housing) 302. In some embodiments, brushes (or brush strands) 303 have integrated sensors in them. For example, the brush strands have one or more sensors integrated to them or within them. In some embodiments, different sensors are placed on different brush strands and brush housing 302.

For example, moisture sensor 304 is integrated at a tip of a brush strand 303; camera lens 305 is coupled to brush housing 302; light source 306 is coupled to brush housing 302 and positioned near camera lens 305; chemical sensor 307 is integrated at the tip of a brush strand 303; and thickness sensor 308 is placed along a brush strand 303. In some embodiments, moisture sensor 304 is same as moisture sensor 204₁; camera lens 305 is like camera lens 205₁, light source 306 is like light source 206₁; chemical sensor 307 is like chemical sensor 207₁; and thickness sensor 308 is like thickness sensor 208, hence their details are not repeated. When brush is applied to the scalp, the sensors and camera (and light source) capture the necessary information to estimate the health of the person.

FIG. 3B illustrates a cross-sectional view 320 of a hair brush of FIG. 3A including one or more sensors used for estimating human health, according to some embodiments of the disclosure. It is pointed out that those elements of FIG. 3B having the same reference numbers (or names) as the elements of any other figure can operate or function in any manner similar to that described, but are not limited to such.

Cross-sectional view 320 illustrates the positions of various sensors at the tips of the hair strands 303. In some embodiments, the thickness sensors can also be placed between the tip of the hair strand 303 and the other end of the hair strand 303 coupled to the main housing 302. In some embodiments, flexible wiring 322 (e.g., copper wiring) connects all sensors to processor or microcontroller 323 (which may be same as processor 223). In some embodiments, main housing 302 includes an antenna coupled to processor 323. Here, antenna 324 may be the same as antenna 224.

In some embodiments, main housing 302 includes a rechargeable battery pack 326 for providing power to processor 323, sensors, and other components (e.g., camera 305, light 306, etc.) of the hair brush. In some embodiments, indicator 325 (e.g., a light indicator or speaker) is coupled to main housing 302 or integrated within main housing 302. In some embodiments, indicator 325 alerts a user of the need to clean the hair brush of stuck hair before it is applied to the scalp.

In some embodiments, the main housing 302 includes vibrator 327. In some embodiments, vibrator 327 is turned on when camera 305 detects hair stuck in the brushes when the hair brush is not in use. For example, when none of the sensor tips are touching the scalp for a threshold duration (e.g., 1 minute) then processor 323 turns on vibrator 327 to shake off any hairs stuck in brushes 303 and/or alerts the user to manually remove the hair from the brushes. In some embodiments, vibrator 327 includes a vibrating motor which may be used to either attempt to clean the bristles of hair or to alert the user to clean the bristles manually.

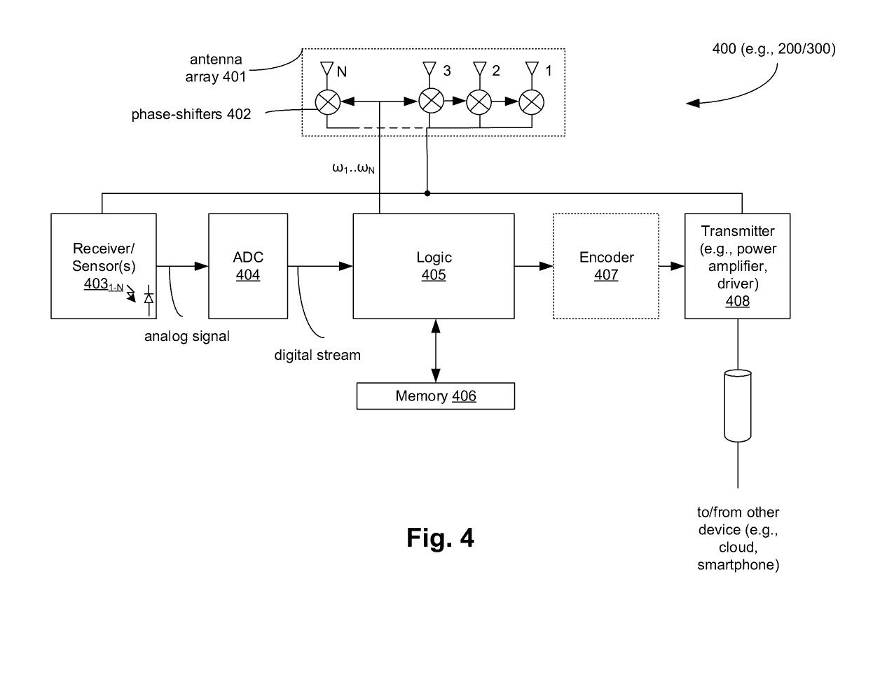

FIG. 4 illustrates a high level architecture 400 of a wearable device (e.g., 200/300), according to some embodiments of the disclosure. It is pointed out that those elements of FIG. 4 having the same reference numbers (or names) as the elements of any other figure can operate or function in any manner similar to that described, but are not limited to such.

In some embodiments, architecture 400 comprises antenna array 401, phase-shifters 402, Receiver/Sensor 403_{1-N} (where ‘N’ is an integer), ADC 404, Logic 405, Memory 406, and Transmitter 408. In some embodiments, architecture 400 includes Encoder 407.

In some embodiments, antenna array 401 may comprise one or more of directional or omnidirectional antennas 1 through ‘N,’ where ‘N’ is an integer, including monopole antennas, dipole antennas, loop antennas, patch antennas, microstrip antennas, coplanar wave antennas, or other types of antennas suitable for transmission of RF signals. In some MIMO embodiments, antenna array 401 are separated to take advantage of spatial diversity.

In some embodiments, phase-shifters 402 are provided to tune the phase of the receiving/transmitting signal. For example, each antenna of antenna array 401 may be coupled to a corresponding phase-shifter, such that phase-shifter 402 coupled to antenna 1 receives phase input on, phase-shifter 402 coupled to antenna 2 receives phase input ω2, and so on. Any known phase-shifter may be used for phase-shifters 402.

In some embodiments, Receiver/Sensors 403_{1-N} (collectively also referred to as 403) detect the received signal and amplifies it to generate an analog signal (where ‘N’ is an integer). An analog signal is any continuous signal for which the time varying feature (variable) of the signal is a representation of some other time varying quantity (i.e., analogous to another time varying signal). For example, Receiver 403 receives sensor data in analog signal form collected from sensors (e.g., 304, 305, 306, 307, 308, 321, and other sensors described here). Here, Receiver/Sensors 403_{1-N} may represent a collection of sensors or receivers for the sensors. In some embodiments, Receiver/Sensor 403 comprises a Low Noise Amplifier (LNA).

In some embodiments, sensors (e.g., 304, 305, 306, 307, 308, 321, and other sensors described here) are digital sensors. In one such embodiment, ADC 404 may not be needed. In some embodiments, sensors (e.g., 304, 305, 306, 307, 308, 321, and other sensors described here) are mix of analog and digital sensors. For example, a light sensor connected to a transistor can directly produce a digital signal, and this light sensor may be used to shine light for the camera and/or for the thickness sensor(s).

In some embodiments, each sensor operates in different frequency channel to allow simultaneous reception by Receiver 403 of sensor data from multiple sensors. Alternatively, in some embodiments, time-sharing can be coordinated between the sensors operating in the same frequency channel. In some embodiments, Receiver 403 receives sensor data through interconnect fabric 322 (e.g., I2C compliant interconnect). In some embodiments, Receiver 403 receives sensor data wirelessly via antenna array 401. As such, Receiver 403 is capable of receiving data through wired and/or wireless means, in accordance with some embodiments.

In some embodiments, the analog signal from Receiver 403 is converted into a digital stream by ADC 404. A digital signal or stream is a physical signal that is a representation of a sequence of discrete values (i.e., a quantified discrete-time signal), for example of an arbitrary bit stream. Any suitable low power ADC may be used to implement ADC 404. For example, ADC 404 is one of: direct-conversion ADC (for flash ADC), successive-approximation ADC, ramp-compare ADC, Wilkinson ADC, integrating ADC, delta-encoded ADC or counter-ramp, pipeline ADC (also called subranging quantizer), sigma-delta ADC (also known as a delta-sigma ADC), time-interleaved ADC, ADC with intermediate FM stage, or time-stretch ADC.

In some embodiments, the digital stream is received by Logic 405 and processed. In some embodiments, Logic 405 (e.g., a Finite State Machine) is a low power logic. In some embodiments, Logic 405 is a low power processor such Intel Curie® designed and manufactured by Intel Corporation of Santa Clara Calif. In some embodiments, Processor 405 is Digital Signal Processor (DSP), an Application Specific Integrated Circuit (ASIC), a general purpose Central Processing Unit (CPU), or any other low power processor that can be integrated into a wearable device. In some embodiments, Logic 405 executes a real-time operating system (RTOS). A RTOS is an OS which is to serve real-time applications and to process data as it comes in, typically without buffering delays.

For example, RTOS is capable of processing sensor data in real-time. In some embodiments, RTOS is operable to perform at least one of: cooperative scheduling, preemptive scheduling (e.g., rate-monotonic scheduling, round-robin scheduling, fixed priority pre-emptive scheduling, fixed-priority scheduling with deferred preemption, fixed-priority non-preemptive scheduling, critical section preemptive scheduling, static time scheduling, etc.), easiest deadline first approach, stochastic digraphs with multi-threaded graph traversal, etc.

In some embodiments, Logic 405 includes sensor drivers for communicating with sensors 403_{1-N} (e.g., 304, 305, 306, 307, 308, 321, and any other sensors described here). In some embodiments, these sensor drivers can be software, firmware, or hardware. In some embodiments, Logic 405 is capable of communicating with derived or virtual sensors. In some embodiments, Logic 405 is operable to perform various algorithms to process sensor data.

For example, Logic 405 can perform basic digital processing (e.g., digital filtering, peak detection, etc.) of sensor data. In some embodiments, Logic 405 stores collected data from sensors in Memory 406. In some embodiments, Memory 406 is level-1 cache. In some embodiments, Memory 406 is a non-volatile memory (e.g., NAND flash memory, magnetic random access memory (MRAM), etc.).

In some embodiments, the output of Logic 405 is encoded by Encoder 407 before it is sent to Transmitter 408. One purpose of encoding is to reduce power dissipation in communicating logic such as Transmitter 408. Encoding the data can also secure the output of sensors from malicious hacks. Any known encoding scheme may be used by Encoder 407. In some embodiments, data processed by Logic 405 is directly provided to Transmitter 408 (i.e., data is not encoded by Encoder 407).

In some embodiments, Transmitter 408 may use any known transmitting scheme. In some embodiments, Transmitter 408 is compliant with WiGig transmission standard (i.e., IEEE 802.11ad transmitting standard). In some embodiments, Transmitter 408 uses Bluetooth technology to transmit data to another device. In some embodiments, Transmitter 408 uses Wi-Fi technology to transmit data to another device. In some embodiments, Transmitter 408 uses WLAN transmissions in accordance with one or more WLAN standards based on CSMA/CD such as those promulgated by the IEEE. In some embodiments, Transmitter 408 may use Long Term Evolution (LTE) compliant transmission mechanisms.

Any suitable low power transmitter may be used for implementing Transmitter 408 (e.g., a transmitter having low power amplifier driver). In some embodiments, Transmitter 408 converts the encoded data to an analog RF signal which is then transmitted by antenna array 401 to Processor 405. In other embodiments, other forms of wireless transmissions may be used by Transmitter 408.

In some embodiments, Transmitter 408 includes a digital-to-analog converter (DAC) (not shown) to convert the encoded probe request and/or probe response messages into analog signal(s) for transmission. In some embodiments, the DAC is a pulse-width modulator (PWM). In some embodiments, the DAC is an oversampling DAC or interpolating DAC such as sigma-delta DAC. In other embodiments, other types of low power DACs may be used. For example, the DAC of Transmitter 408 is one of switched resistor DAC, switched current source DAC, switched capacitor DAC, R-2R binary weighted DAC, Successive-Approximation or Cyclic DAC, thermometer-coded DAC, etc. The output the DAC is an analog signal which is amplified and then transmitted to antenna array 401 to the other device(s), according to some embodiments.

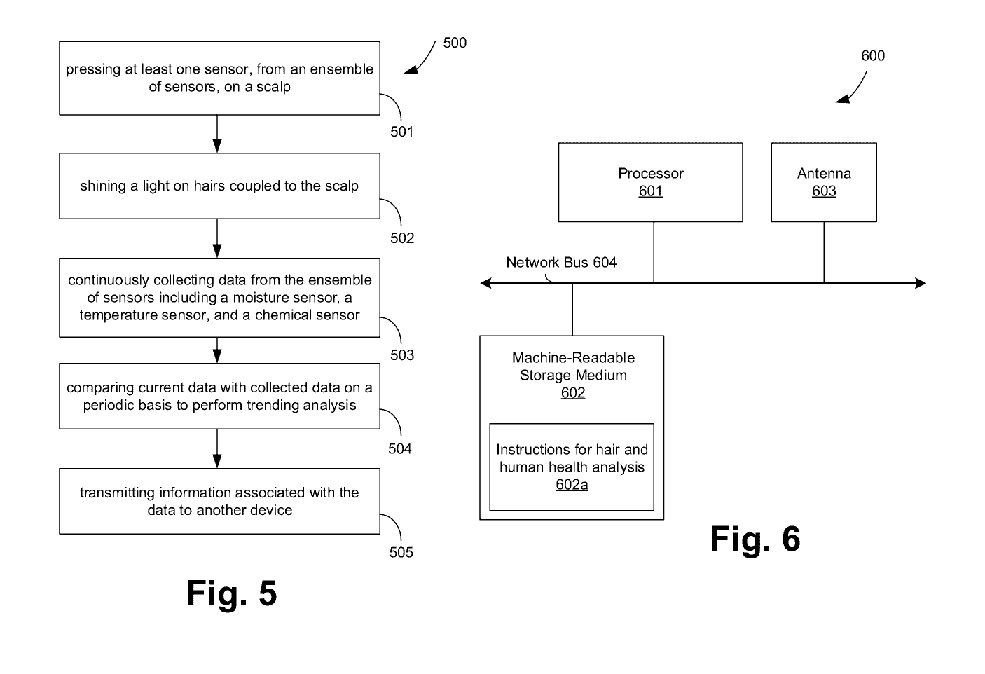

FIG. 5 illustrates flowchart 500 of a method for monitoring hair parameters for estimating human health, according to some embodiments of the disclosure. It is pointed out that those elements of FIG. 5 having the same reference numbers (or names) as the elements of any other figure can operate or function in any manner similar to that described, but are not limited to such.

Although the blocks in the flowchart with reference to FIG. 5 are shown in a particular order, the order of the actions can be modified. Thus, the illustrated embodiments can be performed in a different order, and some actions/blocks may be performed in parallel. Some of the blocks and/or operations listed in FIG. 5 are optional in accordance with certain embodiments. The numbering of the blocks presented is for the sake of clarity and is not intended to prescribe an order of operations in which the various blocks must occur. Additionally, operations from the various flows may be utilized in a variety of combinations.

At block 501, a wearable device (e.g., inner covering 105 of a helmet or cap/hat, hair band 201, hair brush 300, legs of a pair of spectacles, etc.) is pressed on the scalp of a person. Once the sensors are in touch with the scalp they can begin gathering data (e.g., scalp moisture, hair moisture, scalp condition, hair thickness, hair density, etc.).

At block 502, light 306 is turned on for camera 305 to take close-up pictures of the hair and scalp. At block 503, processor 323 continuously collects data from the ensemble of sensors. In some embodiments, processor 323 analyzes the collected data and/or stores the data for future processing.

For example, at block 504, processor 323 compares current data collected from the ensemble of sensors and compares with data stored in the memory (e.g., memory 406) to perform trending analysis (e.g., level of moisture over time, temperature over time, etc.). In some embodiments, at block 505, processor 323 transmitted information associated with the data (e.g., sensor data, initial analysis information, etc.) to another device (e.g., cloud, smart-device) for further processing and analysis.

FIG. 6 illustrates a part of system of FIG. 4 with a machine readable storage medium (or media) having instructions for hair and human health analysis, according to some embodiments of the disclosure. It is pointed out that those elements of FIG. 6 having the same reference numbers (or names) as the elements of any other figure can operate or function in any manner similar to that described, but are not limited to such.

In some embodiments, system 600 (or sensing system 400) comprises a low power Processor 601 (same as Processor 405). In some embodiments, Processor 601 is a Digital Signal Processor (DSP), an Application Specific Integrated Circuit (ASIC), a general purpose Central Processing Unit (CPU), or a low power logic implementing a simple finite state machine to perform the method of flowchart 500 and/or various embodiments, etc. In some embodiments, system 600 comprises Machine-Readable Storage Medium 602 (also referred to as tangible machine readable medium), Antenna 603, and Network Bus 604.

In some embodiments, the various logic blocks of system 600 are coupled together via Network Bus 604. Any suitable protocol may be used to implement Network Bus 604. In some embodiments, Machine-Readable Storage Medium 602 includes Instructions 602a (also referred to as the program software code/instructions) for calculating or measuring distance and relative orientation of a device with reference to another device as described with reference to various embodiments and flowchart.

Program software code/instructions 602a associated with flowchart 500 (and/or various embodiments) and executed to implement embodiments of the disclosed subject matter may be implemented as part of an operating system or a specific application, component, program, object, module, routine, or other sequence of instructions or organization of sequences of instructions referred to as “program software code/instructions,” “operating system program software code/instructions,” “application program software code/instructions,” or simply “software” or firmware embedded in processor. In some embodiments, the program software code/instructions associated with flowchart 500 (and/or various embodiments) are executed by system 600.

In some embodiments, the program software code/instructions 602a associated with flowchart 500 (and/or various embodiments) are stored in a computer executable storage medium 602 and executed by Processor 601. Here, computer executable storage medium 602 is a tangible machine readable medium that can be used to store program software code/instructions and data that, when executed by a computing device, causes one or more processors (e.g., Processor 601) to perform a method(s) as may be recited in one or more accompanying claims directed to the disclosed subject matter.

The tangible machine readable medium 602 may include storage of the executable software program code/instructions 602a and data in various tangible locations, including for example ROM, volatile RAM, non-volatile memory and/or cache and/or other tangible memory as referenced in the present application. Portions of this program software code/instructions 602a and/or data may be stored in any one of these storage and memory devices. Further, the program software code/instructions can be obtained from other storage, including, e.g., through centralized servers or peer to peer networks and the like, including the Internet. Different portions of the software program code/instructions and data can be obtained at different times and in different communication sessions or in the same communication session.

The software program code/instructions 602a (associated with flowchart 600 and other embodiments) and data can be obtained in their entirety prior to the execution of a respective software program or application by the computing device. Alternatively, portions of the software program code/instructions 602a and data can be obtained dynamically, e.g., just in time, when needed for execution. Alternatively, some combination of these ways of obtaining the software program code/instructions 602a and data may occur, e.g., for different applications, components, programs, objects, modules, routines or other sequences of instructions or organization of sequences of instructions, by way of example. Thus, it is not required that the data and instructions be on a tangible machine readable medium in entirety at a particular instance of time.

Examples of tangible computer-readable media 602 include but are not limited to recordable and non-recordable type media such as volatile and non-volatile memory devices, read only memory (ROM), random access memory (RAM), flash memory devices, floppy and other removable disks, magnetic storage media, optical storage media (e.g., Compact Disk Read-Only Memory (CD ROMS), Digital Versatile Disks (DVDs), etc.), among others. The software program code/instructions may be temporarily stored in digital tangible communication links while implementing electrical, optical, acoustical or other forms of propagating signals, such as carrier waves, infrared signals, digital signals, etc. through such tangible communication links.

In general, tangible machine readable medium 602 includes any tangible mechanism that provides (i.e., stores and/or transmits in digital form, e.g., data packets) information in a form accessible by a machine (i.e., a computing device), which may be included, e.g., in a communication device, a computing device, a network device, a personal digital assistant, a manufacturing tool, a mobile communication device, whether or not able to download and run applications and subsidized applications from the communication network, such as the Internet, e.g., an iPhone®, Galaxy®, Blackberry® Droid®, or the like, or any other device including a computing device. In one embodiment, processor-based system is in a form of or included within a PDA (personal digital assistant), a cellular phone, a notebook computer, a tablet, a game console, a set top box, an embedded system, a TV (television), a personal desktop computer, etc. Alternatively, the traditional communication applications and subsidized application(s) may be used in some embodiments of the disclosed subject matter.

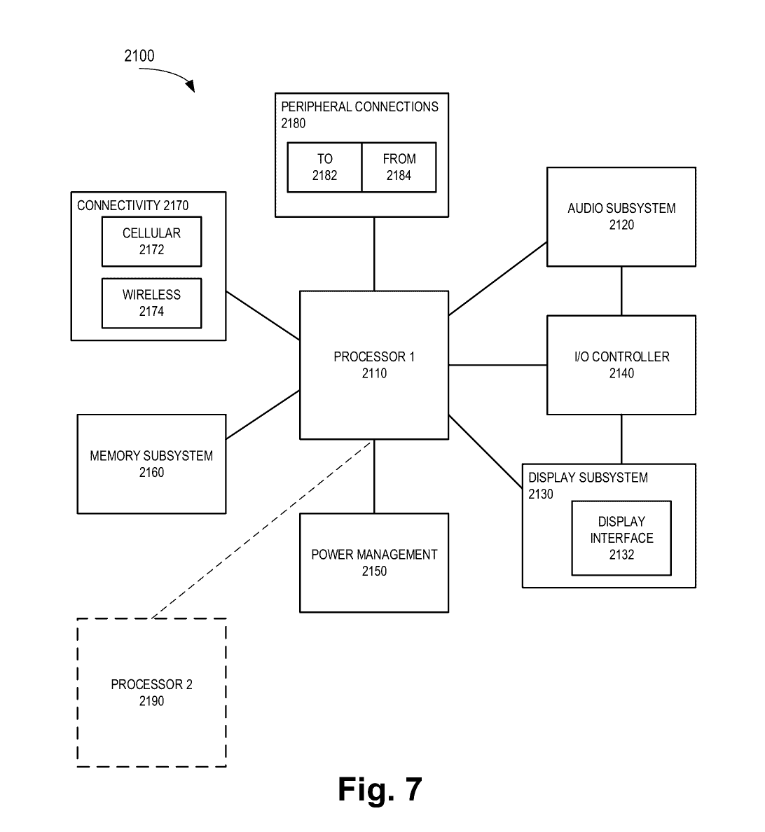

FIG. 7 illustrates a smart device or a computer system or a SoC (System-on-Chip) for processing data collected by one of more sensors for estimating human health and/or changes to human health, according to some embodiments. It is pointed out that those elements of FIG. 7 having the same reference numbers (or names) as the elements of any other figure can operate or function in any manner similar to that described, but are not limited to such.

FIG. 7 illustrates a block diagram of an embodiment of a mobile device in which flat surface interface connectors could be used. In some embodiments, computing device 2100 represents a mobile computing device, such as a computing tablet, a mobile phone or smart-phone, a wireless-enabled e-reader, or other wireless mobile device. It will be understood that certain components are shown generally, and not all components of such a device are shown in computing device 2100.

In some embodiments, computing device 2100 includes a first processor 2110. The various embodiments of the present disclosure may also comprise a network interface within 2170 such as a wireless interface so that a system embodiment may be incorporated into a wireless device, for example, cell phone or personal digital assistant.

In one embodiment, processor 2110 (and/or processor 2190) can include one or more physical devices, such as microprocessors, application processors, microcontrollers, programmable logic devices, or other processing means. The processing operations performed by processor 2110 include the execution of an operating platform or operating system on which applications and/or device functions are executed. The processing operations include operations related to I/O (input/output) with a human user or with other devices, operations related to power management, and/or operations related to connecting the computing device 2100 to another device. The processing operations may also include operations related to audio I/O and/or display M.

In one embodiment, computing device 2100 includes audio subsystem 2120, which represents hardware (e.g., audio hardware and audio circuits) and software (e.g., drivers, codecs) components associated with providing audio functions to the computing device. Audio functions can include speaker and/or headphone output, as well as microphone input. Devices for such functions can be integrated into computing device 2100, or connected to the computing device 2100. In one embodiment, a user interacts with the computing device 2100 by providing audio commands that are received and processed by processor 2110.

Display subsystem 2130 represents hardware (e.g., display devices) and software (e.g., drivers) components that provide a visual and/or tactile display for a user to interact with the computing device 2100. Display subsystem 2130 includes display interface 2132, which includes the particular screen or hardware device used to provide a display to a user. In one embodiment, display interface 2132 includes logic separate from processor 2110 to perform at least some processing related to the display. In one embodiment, display subsystem 2130 includes a touch screen (or touch pad) device that provides both output and input to a user.

I/O controller 2140 represents hardware devices and software components related to interaction with a user. I/O controller 2140 is operable to manage hardware that is part of audio subsystem 2120 and/or display subsystem 2130. Additionally, I/O controller 2140 illustrates a connection point for additional devices that connect to computing device 2100 through which a user might interact with the system. For example, devices that can be attached to the computing device 2100 might include microphone devices, speaker or stereo systems, video systems or other display devices, keyboard or keypad devices, or other I/O devices for use with specific applications such as card readers or other devices.

As mentioned above, I/O controller 2140 can interact with audio subsystem 2120 and/or display subsystem 2130. For example, input through a microphone or other audio device can provide input or commands for one or more applications or functions of the computing device 2100. Additionally, audio output can be provided instead of, or in addition to display output. In another example, if display subsystem 2130 includes a touch screen, the display device also acts as an input device, which can be at least partially managed by I/O controller 2140. There can also be additional buttons or switches on the computing device 2100 to provide I/O functions managed by I/O controller 2140.

In one embodiment, I/O controller 2140 manages devices such as accelerometers, cameras, light sensors or other environmental sensors, or other hardware that can be included in the computing device 2100. The input can be part of direct user interaction, as well as providing environmental input to the system to influence its operations (such as filtering for noise, adjusting displays for brightness detection, applying a flash for a camera, or other features).

In one embodiment, computing device 2100 includes power management 2150 that manages battery power usage, charging of the battery, and features related to power saving operation. Memory subsystem 2160 includes memory devices for storing information in computing device 2100. Memory can include nonvolatile (state does not change if power to the memory device is interrupted) and/or volatile (state is indeterminate if power to the memory device is interrupted) memory devices. Memory subsystem 2160 can store application data, user data, music, photos, documents, or other data, as well as system data (whether long-term or temporary) related to the execution of the applications and functions of the computing device 2100.

Elements of embodiments are also provided as a machine-readable medium (e.g., memory 2160) for storing the computer-executable instructions. The machine-readable medium (e.g., memory 2160) may include, but is not limited to, flash memory, optical disks, CD-ROMs, DVD ROMs, RAMs, EPROMs, EEPROMs, magnetic or optical cards, phase change memory (PCM), or other types of machine-readable media suitable for storing electronic or computer-executable instructions. For example, embodiments of the disclosure may be downloaded as a computer program (e.g., BIOS) which may be transferred from a remote computer (e.g., a server) to a requesting computer (e.g., a client) by way of data signals via a communication link (e.g., a modem or network connection).

Connectivity 2170 includes hardware devices (e.g., wireless and/or wired connectors and communication hardware) and software components (e.g., drivers, protocol stacks) to enable the computing device 2100 to communicate with external devices. The computing device 2100 could be separate devices, such as other computing devices, wireless access points or base stations, as well as peripherals such as headsets, printers, or other devices.

Connectivity 2170 can include multiple different types of connectivity. To generalize, the computing device 2100 is illustrated with cellular connectivity 2172 and wireless connectivity 2174. Cellular connectivity 2172 refers generally to cellular network connectivity provided by wireless carriers, such as provided via GSM (global system for mobile communications) or variations or derivatives, CDMA (code division multiple access) or variations or derivatives, TDM (time division multiplexing) or variations or derivatives, or other cellular service standards. Wireless connectivity (or wireless interface) 2174 refers to wireless connectivity that is not cellular, and can include personal area networks (such as Bluetooth, Near Field, etc.), local area networks (such as Wi-Fi), and/or wide area networks (such as WiMax), or other wireless communication.

Peripheral connections 2180 include hardware interfaces and connectors, as well as software components (e.g., drivers, protocol stacks) to make peripheral connections. It will be understood that the computing device 2100 could both be a peripheral device (“to” 2182) to other computing devices, as well as have peripheral devices (“from” 2184) connected to it. The computing device 2100 commonly has a “docking” connector to connect to other computing devices for purposes such as managing (e.g., downloading and/or uploading, changing, synchronizing) content on computing device 2100. Additionally, a docking connector can allow computing device 2100 to connect to certain peripherals that allow the computing device 2100 to control content output, for example, to audiovisual or other systems.

In addition to a proprietary docking connector or other proprietary connection hardware, the computing device 2100 can make peripheral connections 2180 via common or standards-based connectors. Common types can include a Universal Serial Bus (USB) connector (which can include any of a number of different hardware interfaces), DisplayPort including MiniDisplayPort (MDP), High Definition Multimedia Interface (HDMI), Firewire, or other types.

Reference in the specification to “an embodiment,” “one embodiment,” “some embodiments,” or “other embodiments” means that a particular feature, structure, or characteristic described in connection with the embodiments is included in at least some embodiments, but not necessarily all embodiments. The various appearances of “an embodiment,” “one embodiment,” or “some embodiments” are not necessarily all referring to the same embodiments. If the specification states a component, feature, structure, or characteristic “may,” “might,” or “could” be included, that particular component, feature, structure, or characteristic is not required to be included. If the specification or claim refers to “a” or “an” element, that does not mean there is only one of the elements. If the specification or claims refer to “an additional” element, that does not preclude there being more than one of the additional element.

Furthermore, the particular features, structures, functions, or characteristics may be combined in any suitable manner in one or more embodiments. For example, a first embodiment may be combined with a second embodiment anywhere the particular features, structures, functions, or characteristics associated with the two embodiments are not mutually exclusive

While the disclosure has been described in conjunction with specific embodiments thereof, many alternatives, modifications and variations of such embodiments will be apparent to those of ordinary skill in the art in light of the foregoing description. The embodiments of the disclosure are intended to embrace all such alternatives, modifications, and variations as to fall within the broad scope of the appended claims.

In addition, well known power/ground connections to integrated circuit (IC) chips and other components may or may not be shown within the presented figures, for simplicity of illustration and discussion, and so as not to obscure the disclosure. Further, arrangements may be shown in block diagram form in order to avoid obscuring the disclosure, and also in view of the fact that specifics with respect to implementation of such block diagram arrangements are highly dependent upon the platform within which the present disclosure is to be implemented (i.e., such specifics should be well within purview of one skilled in the art). Where specific details (e.g., circuits) are set forth in order to describe example embodiments of the disclosure, it should be apparent to one skilled in the art that the disclosure can be practiced without, or with variation of, these specific details. The description is thus to be regarded as illustrative instead of limiting.

The following examples pertain to further embodiments. Specifics in the examples may be used anywhere in one or more embodiments. All optional features of the apparatus described herein may also be implemented with respect to a method or process.

For example, a wearable apparatus which comprises: an ensemble of sensors including a moisture sensor to sense moisture content of hair, the moisture sensor positioned in a first housing to be in contact with the hair; and a processor to receive data associated with sensed moisture content and to transmit information related to the received data to another device, wherein the processor is positioned in a second housing different from the first housing. In some embodiments, the processor includes a transmitter to wirelessly transmit the information related to the received data to the other device.

In some embodiments, the ensemble of sensors comprises: a camera; and an actuator to provide light to the hair for the camera to take a picture of the hair. In some embodiments, the actuator is operable to provide at least one of: polarized light; ultraviolet light; or florescent light. In some embodiments, the ensemble of sensors comprises a first chemical sensor which is configured to sense one kind of protein associated with the hair.

In some embodiments, the ensemble of sensors comprises a second chemical sensor which is configured to sense mineral content associated with the hair. In some embodiments, the ensemble of sensors comprises a temperature sensor to sense temperature of a scalp. In some embodiments, the processor is operable to continuously collect data from the ensemble of sensors. In some embodiments, the other device is at least one of: cloud, smartphone, tablet, or personal computer. In some embodiments, the ensemble of sensors are positioned in one of: a hair brush, a hair clip, a hair band, legs of a pair of spectacles, comb, a cap, or a hat.

In another example, an apparatus is provided which comprises: a housing; a plurality of bristles originating from the housing which is to hold the plurality of bristles, wherein at least one of the bristles includes a moisture sensor positioned at a tip of the at least one of the bristles; a camera lens coupled to the housing, the camera lens is to face the tip of the at least one of the bristles; and a light source coupled the housing to provide light for the camera lens. In some embodiments, the apparatus comprises a thickness sensor coupled to the at least one of the bristles away from the tip.

In some embodiments, the light source is operable to provide at least one of: polarized light; ultraviolet light; or florescent light. In some embodiments, the camera lens is a macro lens. In some embodiments, the apparatus comprises a vibrating motor to vibrate the apparatus to alert a user to remove any hair from the plurality of bristles. In some embodiments, the apparatus comprises a speaker or a light indicator to indicate that hair is stuck in the plurality of bristles.

In another example, a method is provided which comprises: pressing at least one sensor, from an ensemble of sensors, on a scalp; shining a light on hairs coupled to the scalp; continuously collecting data from the ensemble of sensors including a moisture sensor, a temperature sensor, and a chemical sensor; and transmitting information associated with the data to another device. In some embodiments, the method comprises: storing the collected data in a memory; and comparing current data with collected data on a periodic basis to perform trending analysis.

In some embodiments, the method comprises indicating an alert when hair is stuck to at least one of the sensors of the ensemble. In some embodiments, the transmitted information is received by the other device, and wherein the other device is to analyze the transmitted information and to estimate hair parameters including at least one of: dryness of hair, dryness of the scalp, thickness of the hair, hair color change, mineral content of hair, temperature of the scalp, and hair density.

In another example, an apparatus is provided which comprises: means for pressing at least one sensor, from an ensemble of sensors, on a scalp; means for shining a light on hairs coupled to the scalp; means for continuously collecting data from the ensemble of sensors including a moisture sensor, a temperature sensor, and a chemical sensor; and means for transmitting information associated with the data to another device. In some embodiments, the apparatus comprises: means for storing the collected data in a memory; and means for comparing current data with collected data on a periodic basis to perform trending analysis.

In some embodiments, the apparatus comprises: means for indicating an alert when hair is stuck to at least one of the sensors of the ensemble. In some embodiments, the transmitted information is received by the other device, and wherein the other device is to analyze the transmitted information and to estimate hair parameters including at least one of: dryness of hair, dryness of the scalp, thickness of the hair, hair color change, mineral content of hair, temperature of the scalp, and hair density.

An abstract is provided that will allow the reader to ascertain the nature and gist of the technical disclosure. The abstract is submitted with the understanding that it will not be used to limit the scope or meaning of the claims. The following claims are hereby incorporated into the detailed description, with each claim standing on its own as a separate embodiment.

We claim:

1. A wearable apparatus comprising:
an ensemble of sensors including a moisture sensor to sense moisture content of hair, the moisture sensor positioned in a first housing to be in contact with the hair; and
a processor to receive data associated with sensed moisture content and to transmit information related to the received data to another device, wherein the processor is positioned in a second housing different from the first housing.

2. The wearable apparatus of claim 1, wherein the processor includes a transmitter to wirelessly transmit the information related to the received data to the other device.

3. The wearable apparatus of claim 1, wherein the ensemble of sensors comprises:
a camera; and
an actuator to provide light to the hair for the camera to take a picture of the hair.

4. The wearable apparatus of claim 3, wherein the actuator is operable to provide at least one of:
polarized light;
ultraviolet light; or
florescent light.

5. The wearable apparatus of claim 3, wherein the ensemble of sensors comprises a first chemical sensor which is configured to sense one kind of protein associated with the hair.

6. The wearable apparatus of claim 5, wherein the ensemble of sensors comprises a second chemical sensor which is configured to sense mineral content associated with the hair.

7. The wearable apparatus of claim 6, wherein the ensemble of sensors comprises a temperature sensor to sense temperature of a scalp.

8. The wearable apparatus of claim 7, wherein the processor is operable to continuously collect data from the ensemble of sensors.

9. The wearable apparatus of claim 1, wherein the other device is at least one of: cloud, smartphone, tablet, or personal computer.

10. The wearable apparatus of claim 1, wherein the ensemble of sensors are positioned in one of: a hair brush, a hair clip, a hair band, legs of a pair of spectacles, comb, a cap, or a hat.

11. An apparatus comprising:
a housing;
a plurality of bristles originating from the housing which is to hold the plurality of bristles, wherein at least one of the bristles includes a moisture sensor positioned at a tip of the at least one of the bristles;
a camera lens coupled to the housing, the camera lens is to face the tip of the at least one of the bristles; and
a light source coupled the housing to provide light for the camera lens.

12. The apparatus of claim 11 comprises a thickness sensor coupled to the at least one of the bristles away from the tip.

13. The apparatus of claim 11, wherein the light source is operable to provide at least one of:
polarized light;
ultraviolet light; or
florescent light.

14. The apparatus of claim 11, wherein the camera lens is a macro lens.

15. The apparatus of claim 11 comprises a vibrating motor to vibrate the apparatus to alert a user to remove any hair from the plurality of bristles.

16. The apparatus of claim 11 comprises a speaker or a light indicator to indicate that hair is stuck in the plurality of bristles.

17. A method comprises:
pressing at least one sensor, from an ensemble of sensors, on a scalp;
shining a light on hairs coupled to the scalp;
continuously collecting data from the ensemble of sensors including a moisture sensor, a temperature sensor, and a chemical sensor; and
transmitting information associated with the data to another device.

18. The method of claim 17 comprises:
storing the collected data in a memory; and
comparing current data with collected data on a periodic basis to perform trending analysis.

19. The method of claim 17 comprises indicating an alert when hair is stuck to at least one of the sensors of the ensemble.

20. The method of claim 17, wherein the transmitted information is received by the other device, and wherein the other device is to analyze the transmitted information and to estimate hair parameters including at least one of: dryness of hair, dryness of the scalp, thickness of the hair, hair color change, mineral content of hair, temperature of the scalp, and hair density.
